(19) United States
(12) Patent Application Publication
(10) Pub. No.: US 2026/0174333 A1
(43) Pub. Date: Jun. 25, 2026

(54) WEARABLE USER DEVICE, COMPUTER PROGRAM, METHOD, AND APPARATUS FOR NON-INVASIVE MEASUREMENT OF FLUIDS IN LIVING TISSUE

(71) Applicant: Sony Group Corporation, Tokyo (JP)

(72) Inventors: Serkan ERYILMAZ, Stuttgart (DE); Daniel Felipe Ardila PALOMINO, Stuttgart (DE); Gerd SPALINK, Stuttgart (DE); Hugo EMBRECHTS, Stuttgart (DE); Gonzalo BAILADOR DEL POZO, Stuttgart (DE); Anja PFLUG, Stuttgart (DE); Pablo BARROS, Stuttgart (DE)

(73) Assignee: Sony Group Corporation, Tokyo (JP)

(21) Appl. No.: 19/127,422

(22) PCT Filed: Sep. 27, 2023
(86) PCT No.: PCT/EP2023/076803

§ 371 Date: May 6, 2025

(30) Foreign Application Priority Data
Nov. 15, 2022 (EP) 22207562.4

(51) Int. Cl. A61B 5/00; A61B 5/0205; A61B 5/145; A61B 5/1455

(52) U.S. Cl. CPC A61B 5/0059; A61B 5/0205; A61B 5/14532; A61B 5/1455; A61B 5/14551

(57) ABSTRACT

The present disclosure relates to a wearable user device, a computer program, a method, and an apparatus for noninvasive measurement of fluids in living tissue. The apparatus comprises a frequency-modulated continuous wave (FMCW) transmitter configured to emit an FMCW signal for reflections of the FMCW signal on a body fluid, a receiver configured to receive the reflections of the FMCW signal, and a processing circuit configured to obtain information on the body fluid based on the reflections of the FMCW signal.

### FIELD

The present disclosure relates to a wearable user device, a computer program, a method, and an apparatus for non-invasive measurement of fluids in living tissue. In particular, but not exclusively, embodiments of the present disclosure relate to a concept for laser doppler flowmetry, LDF, of blood.

### BACKGROUND

Laser Doppler Flowmetry (LDF) is a non-invasive technique to measure blood flow in tissues, so that relevant biomarkers can be estimated. A fundamental principle of the LDF technique is to measure a frequency shift of reflections of light on the tissue of interest. The frequency shift is a consequence of the movement of red blood cells in the vessels of the tissue. So, LDF allows to extract information from the movement of red blood cells. Even though LDF has multiple advantages with respect to other techniques for blood flow monitoring (e.g., ultrasonic or contrast methods), it has some critical drawbacks. Two of the main limitations of using LDF in practical applications are motion artifact noise and lack of depth disambiguation. Motion artifacts are an inherent problem with the LDF measurement principle. These artifacts refer to the noise created by the movement of the tissue with respect to a measurement device and movement of the fiber-optic probes. The noise resulting from the relative movement of the tissue and the measurement device is especially relevant for applications in wearable devices and patients that tend to move during measurements like infants. Such motion artifacts degrade a measurement quality and can affect results of blood flow measurement.

One approach to remove the motion noise coming from the relative movement of the tissue and the measurement device suggests the use of polarization techniques. Such an approach uses polarized light to illuminate the tissue, so that specular reflections on the surface of the skin can be filtered out and thus the motion noise can be reduced significantly. However, filtering out the specular reflections affects a signal to noise ratio since reflections on the red blood cells could be removed as well.

Hence, there is a demand of an improved concept of non-invasive measurements of fluids in living tissue.

### SUMMARY

This demand is met by the subject-matter of the appended independent claims. Advantageous embodiments are addressed by the dependent claims.

Some aspects of the present disclosure relate to an apparatus for non-invasive measurement of fluids in living tissue, the apparatus comprising a frequency-modulated continuous wave (FMCW) transmitter configured to emit an FMCW signal for reflections of the FMCW signal on a body fluid, a receiver configured to receive the reflections of the FMCW signal, and a processing circuit configured to obtain information on the body fluid based on the reflections of the FMCW signal. As a skilled person having benefit from the present disclosure will appreciate, the reflections of the FMCW signal not only allows to determine a Doppler shift but also a travelling time of the FMCW signal and the reflections. Hence, the FMCW signal allows to determine a target distance for a more accurate investigation of the body fluid.

In some embodiments, the processing circuit may be configured to obtain the information on the body fluid based on a comparison of the FMCW signal and its reflections.

The present approach, e.g., may be used in flowmetry applications. Accordingly, the information on the body fluid may comprise information on a flow of the body fluid. In practice, the information on the flow, e.g., comprises information on a flow velocity of the flow. Optionally, the information on the flow may also comprise information on a flow rate of the flow. Such information on the velocity and/or flow rate then may allow to determine biomarkers.

In some embodiments, the information on the body fluid may comprise information on a depth of the body fluid in the living tissue. The information on the depth, e.g., allows to distinguish between information on the body fluid by its depth, as explained in more detail later.

According to an example, the processing circuit may be configured to filter measurement data indicative of the reflections using the information on the depth. In doing so, e.g., unwanted noise resulting from reflections on a skin surface may be filtered out to enhance the measurement data, as laid out later in more detail.

For example, the processing circuit may be configured to obtain information on a vessel of the body fluid from the reflections of the FMCW signal. In practice, the information on the depth of the body fluid allows to distinguish between different vessels and, thus, to characterize a specific vessel and/or the flow of body fluid therein. In applications, the vessel can be any vessel containing and/or conveying a body fluid. The vessel, for example, is a blood vessel or a lymphatic vessel.

In practice, the body fluid may comprise blood. In this case, the information may comprise information on a pulse rate, a blood pressure, and/or an oxygen saturation (level) of the blood.

The skilled person having benefit from the present disclosure will appreciate that the reflections of the FMCW signal allow to distinguish between different types of blood vessels. For this, the processing circuit may be configured to extract information on blood flowing through capillaries and/or information on blood flowing through veins and/or arteries from the information on the body fluid. This, e.g., allows to distinguish between capillaries, veins and/or arteries and to evaluate information on blood flowing through capillaries blood flowing through arteries and/or veins separately for a more accurate and/or reliable determination of a physical condition.

Information on the blood flowing through the capillaries, e.g., allows to determine microvascular dynamics. Accordingly, the processing circuitry may be configured to obtain information on microvascular dynamics from the information on the blood flowing through the capillaries.

The information on the blood flowing through veins and/or arteries allows to measure a cardiac activity. Accordingly, the processing circuitry may be configured to obtain information on a cardiac activity from the information on the blood flowing through the veins and/or arteries. The cardiac activity, e.g., includes the activity of the myocardium, the vessels and their musculature, and the neuronal (e.g., sympathetic and parasympathetic) and endocrine control mechanisms (e.g., circulating catecholamines) of the cardiovascular system.

As the skilled person having benefit from the present disclosure will appreciate the FMCW signal may be adapted to different use cases. In particular, the FMCW signal may be adapted for examination at different depths. In practice, the FMCW signal may be adapted to penetrate skin up to 4 millimeters deep. In this way, the signal reaches far enough to examine arteries, veins, and capillaries. For this, parameters of the FMCW signal may be adapted.

According to an example, a carrier frequency of the FMCW signal may be between 150 THz and 600 THz, a chirp bandwidth of the FMWC signal may be between 1 THz and 10 THz, a chirping time of the FMCW signal may be between 5 μs and 100 μs, and/or a chirp repetition interval may be between 10 μs and 200 μs.

The skilled person will appreciate that the present concept can be implemented with different transmitters. In some applications, the FMCW transmitter may comprise a Vertical-Cavity Surface-Emitting Laser (VCSEL).

Another aspect of the present disclosure relates to a wearable user device comprising an apparatus of any one of the preceding claims.

Some aspects of the present disclosure relate to a method for non-invasive measurement of fluids in living tissue, the method comprising receiving information on reflections of a FMCW signal on a body fluid and obtaining information on the body fluid based on the reflections of the FMCW signal.

An aspect of the present disclosure relates to a computer program having a program code for performing a method according to the present disclosure when the program is executed on a processor or a programmable hardware.

### BRIEF DESCRIPTION OF THE FIGURES

Some examples of apparatuses and/or methods will be described in the following by way of example only, and with reference to the accompanying figures, in which

FIG. 1 shows a block diagram schematically illustrating an example of an apparatus for noninvasive measurement of fluids in living tissue;

FIGS. 2a and 2b schematically illustrate a use case of another embodiment of the apparatus;

FIG. 3 shows a block diagram schematically illustrating still another example of the apparatus;

FIG. 4 schematically depicts a measurement of a body fluid according to the present disclosure;

FIGS. 5a and 5b exemplarily illustrate simulated results of the measurement; and

FIG. 6 shows a flow chart schematically illustrating an embodiment of a method for noninvasive measurement of fluids in living tissue.

### DETAILED DESCRIPTION

Some examples are now described in more detail with reference to the enclosed figures. However, other possible examples are not limited to the features of these embodiments described in detail. Other examples may include modifications of the features as well as equivalents and alternatives to the features. Furthermore, the terminology used herein to describe certain examples should not be restrictive of further possible examples.

Throughout the description of the figures same or similar reference numerals refer to same or similar elements and/or features, which may be identical or implemented in a modified form while providing the same or a similar function. The thickness of lines, layers and/or areas in the figures may also be exaggerated for clarification.

When two elements A and B are combined using an “or”, this is to be understood as disclosing all possible combinations, i.e. only A, only B as well as A and B, unless expressly defined otherwise in the individual case. As an alternative wording for the same combinations, “at least one of A and B” or “A and/or B” may be used. This applies equivalently to combinations of more than two elements.

If a singular form, such as “a”, “an” and “the” is used and the use of only a single element is not defined as mandatory either explicitly or implicitly, further examples may also use several elements to implement the same function. If a function is described below as implemented using multiple elements, further examples may implement the same function using a single element or a single processing entity. It is further understood that the terms “include”, “including”, “comprise” and/or “comprising”, when used, describe the presence of the specified features, integers, steps, operations, processes, elements, components and/or a group thereof, but do not exclude the presence or addition of one or more other features, integers, steps, operations, processes, elements, components and/or a group thereof.

Laser Doppler Flowmetry sensors typically use a single color modulation scheme that only provides information on a velocity of the red blood cells and does not allow to determine their depth. One finding of the present disclosure is that information on the depth of body fluids allows to discriminate different structures in the tissue that have specific behaviors, so that a more insightful analysis can be performed. Additionally, by having such depth information available it is possible to focus the analysis of the signals only on a specific section of the tissue that is relevant. In doing so, e.g., reflections coming from a skin surface can be selectively removed. As outlined in more detail with reference to the appended drawings, it is proposed that, to this end, Frequency Modulated Continuous Wave (FMCW) principles are applied to determine doppler and range information simultaneously.

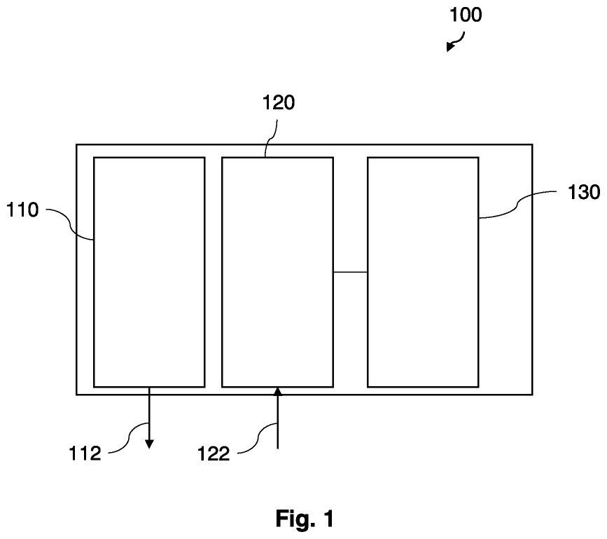

The concept of the present disclosure, e.g., is implemented in an apparatus, as laid out in more detail with reference to FIG. 1.

FIG. 1 shows a block diagram schematically illustrating an example of an apparatus 100 for non-invasive measurement of fluids in living tissue.

The apparatus 100 comprises an FMCW transmitter 110 configured to emit an FMCW signal 112 for reflections 122 of the FMCW signal 112 on a body fluid. The FMCW signal 112 is a signal which varies in frequency through modulation. In practice, the frequency of the FMCW signal 112 varies up and down, e.g., the FMCW signal 112 may oscillate with a predefined period. So, according to the FMCW principle that typically is used in connection with radar systems, instead of transmitting a single wavelength signal, the frequency of the signal is changed according to some time-dependent function providing a certain modulation scheme.

As the skilled person will appreciate, different modulation schemes may be used for the modulation of the FMCW signal 112. The modulation results in a series of recurring frequency changes, also referred to as “chirps”. In practice, the frequency may change linearly. Examples of such schemes, e.g., provide a sawtooth wave, triangle wave, or square wave modulation of the FMCW signal 112. However, in some examples, the frequency may also change non-linearly but, e.g., exponentially. In practice, so-called “up chirps” or “down chirps” may be applied.

The FMCW transmitter 110, e.g., includes a tunable laser. For measurements of the body fluid, the FMCW 112 signal can be such that it penetrates the tissue far enough to reach the body fluid and that the reflections 122 on the body fluid at least partly leave the tissue and are measurable outside the tissue. To this end, a signal power and other signal parameters of the FMCW signal 112 may be adapted accordingly such that the FMCW signal 112 penetrates the tissue far enough and that it is sufficiently reflected by the body fluid. In doing so, the FMCW signal 112 can be adapted to different tissues and/or body fluids.

Further, the apparatus 100 comprises a receiver 120 configured to receive the reflections 122 of the FMCW signal 112. For this, the receive may comprise a photosensitive sensor which is sensitive to the reflections 122 of the FMCW signal 112. The FMCW transmitter 110 and the receiver 120 can be implemented in a monostatic configuration, e.g., in a FMCW transceiver with the capabilities of the described transmitter 110 and receiver 120. As well, the FMCW transmitter 110 and the receiver 120 can be implemented separately in a bistatic configuration.

Also, the apparatus 100 comprises a processing circuit 130 configured to obtain information on the body fluid based on the reflections 122 of the FMCW signal 112. For this, the receiver 120 may provide a measurement signal indicative of the reflections 122 to the processing circuit 130 and the processing circuit 130 determines the information on the body fluid from the measurement signal.

As the skilled person will understand that the reflections 122 of the FMCW signal 112 allow to determine information on a flow of the body fluid from a Doppler shift of the reflections 122 relative to the emitted FMCW signal 112 (based on a radial velocity of the target). In particular, the information on the flow may comprise information on a velocity and/or flow rate of the flow. In order to obtain such information, the processing circuit 130, e.g., compares the FMCW signal 112 and the reflections 122 to determine the Doppler shift. Then, information on a physical condition and/or biomarkers may be determined from the information on the flow. In practice, the body fluid, e.g., is blood and the information on the flow may provide various information on a physical condition and/or activity of a cardiovascular system.

As well, information on a vessel of the body fluid may be obtained from the reflections of the FMCW signal 112. In practice, the information on the vessel, e.g., comprises information on a health state of vessels and/or a vascular disease. In practice, the information on the flow of the body fluid.

Also, the information may comprise information on a pulse rate and/or a blood pressure. The pulse rate, e.g., is obtained from speed and/or range variations indicated by the reflections and the blood pressure, e.g., is obtained from speed information on the blood flow.

As the skilled person will appreciate, the FMCW signal 112 may be also used for absorbance and/or reflectance-based pulse oximetry. Accordingly, the processing circuit 130 may be also configured to determine the blood saturation level from the reflections 122 of the FMCW signal 112.

As well, the modulation of the FMCW signal 112 allows to determine a frequency shift between the modulation of the FMCW signal 112 and the modulation of the received reflections 122 from the comparison of the FMCW signal 112 and the reflections 122. The frequency shift indicates a signal travelling time of the FMCW signal 112 and its reflections 122 and, so, provides information on a distance of the body fluid reflecting the FMCW signal 112. Additionally, a phase shift between chirps of the FMWC signal 112 and the reflections 122 may be determined to obtain a speed of the flow of the body fluid.

So, by comparing the transmitted and received signal it is possible to determine not only flow information (velocity, flow rate, and/or the like) but also range information that, e.g., allows to determine a depth of the body fluid in the tissue. Thus, the modulation of the FMCW signal 112 allows to isolate contributions of the Doppler shift and the time of travel of the FMCW signal 112 and its reflections 122, so that the flow information and depth information can be obtained from the reflections 122. The information on the body fluid, thus, may include particularly information on a depth of the body fluid in the living tissue.

In practice, the range information, e.g., indicates a distance between the apparatus 100 and the body fluid. In scenarios where the apparatus 100 is firmly attached to the skin surface, the depth may (approximately) correspond to the distance (possibly minus a predefined distance of the skin to the FMCW transmitter and/or receiver).

Also, the depth may be determined from a comparison of range information on a distance to the skin (surface) and the body fluid. This, e.g., also allows to determine the depth in scenarios where the apparatus 100 is loosely attached and may move relative to the skin surface.

In applications, the range information may provide several advantages. It, e.g., allows to differentiate between reflections in the tissue by its distance or depth, so that specific areas of interest can be investigated in a targeted or selective manner.

As well, the modulation allows to isolate or filter out noise resulting from relative movements of a skin surface and the apparatus, so that noise is mitigated without compromising the quality of the signal coming from the red blood cells in the tissue.

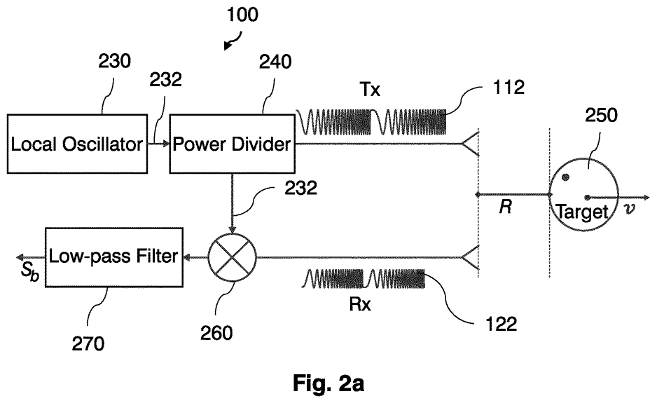

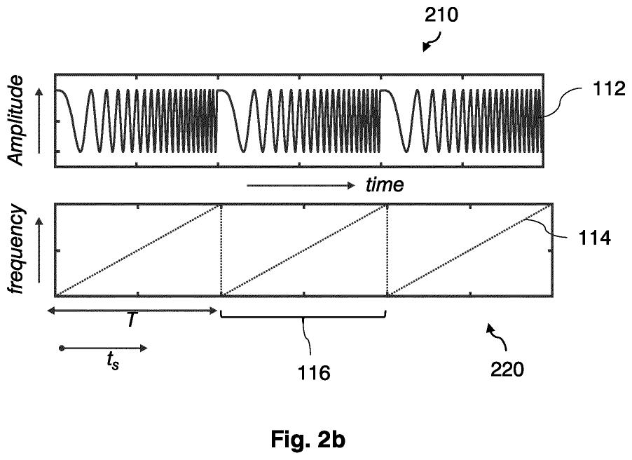

The skilled person will appreciate that the proposed approach can be applied in various use cases. In particular, the proposed approach can be adapted for measurements of any body fluid and/or any living tissue. So, the proposed approach can be adapted to different living beings, e.g., humans or animals. The measurements may be used for medical or other technical purposes such as for sports watches. An exemplary use case is described in greater detail with reference to FIGS. 2a and 2b.

FIG. 2a shows a block diagram schematically illustrating another embodiment of the apparatus 100. As can be seen from FIG. 2a, the apparatus 100, e.g., comprises a local oscillator (LO) 230 providing an oscillator signal 232 for generating the FMCW signal 112. The LO 230 transmits the oscillator signal 232 to a power divider 240 which forwards the oscillator signal 232 to a mixer 260. Also, the oscillator signal 232 serves for generating the FMCW signal 112.

The FMCW signal 112 is then emitted towards a target 250, here, e.g., towards a living tissue including body fluid. In exemplary applications, the apparatus, e.g., may be attached to a person or an animal for investigating the physical condition of the person or animal. For this, the apparatus 100 may be attached to the person or animal. The body fluid that is supposed to be investigated, e.g., is blood. In this case, the FMCW signal 112 may be particularly adapted such that the FMCW signal 112 is reflected by blood. To this end, the FMCW signal 112, e.g., is adapted such that it is reflected by red blood cells. In other cases, the FMCW signal 112 may be adapted to investigate other body fluids such as lymphatic fluid.

The FMCW signal 112, then, is reflected by the blood cells and the reflections 122 of the FMCW signal 112 are sensed by the apparatus. In doing so, a measurement signal indicative of the reflections 122 is generated. The measurement signal, then, is transmitted to the mixer 260. The mixer 260 generates a mixed signal including the oscillator signal 232 and the measurement signal for comparison of the measurement signal with the oscillator signal 232 as reference. The mixed signal, then, is filtered through a low-pass filter 270 to obtain a signal s_{b}. Then, a velocity v of the blood cells and a distance R may be determined from signal s_{b}.

As the skilled person will appreciate, the proposed approach is not limited to a specific frequency modulation but is compatible with various modulation schemes for the FMCW signal 112. For further details, the proposed approach is described in more detail with reference to an exemplary modulation scheme illustrated in FIG. 2b.

FIG. 2b shows a first (upper) diagram 210 depicting a plot of an amplitude of the FMCW signal 112 versus time, wherein the ordinate of diagram 210 indicates the amplitude and the abscissa of diagram 210 indicates the time. Further, FIG. 2b shows a second (lower) diagram 220 depicting a plot 114 of the frequency of the FMCW signal 112, wherein the ordinate of diagram 220 indicates the frequency and the abscissa of diagram 220 indicates the time and is synchronous to the abscissa of diagram 210.

As can be seen from the diagrams 210 and 220, the frequency of the FMCW signal 112 varies with time. In the shown example, the FMWC signal 112, e.g., comprises a series of recurring frequency variations 116, so-called “chirps”, which are repeated periodically, here with period T. In the shown example, a sawtooth modulation is applied. In doing so, the frequency variations 116 comprise a linear ramp of the frequency from a predefined lower frequency to a predefined upper frequency. Here, the frequency of the transmitted signal, e.g., increases linearly from a carrier frequency f_{c} up to f_{c}+BW in time T, where BW represents its bandwidth. Once the upper frequency is reached, the frequency variation 116 is repeated. In the present case, the frequency variation 116 is repeated N times during a predefined measurement frame. To facilitate the mathematical description of the chirping sequence two auxiliar parameters are introduced:
tₛ which denotes the time, also referred to as “fast-time”, from the beginning of a single chirp, and
n which represents a chirp number in the measurement frame. n is also referred to as “slow-time”.

### With Reference to the Above Parameters, Time can be Expressed as Follows:

$$t = n T + {t}_{s} , \qquad ( 1$$

wherein 0≤tₛ≤ST.
The signal s_{b}, then, can be expressed as follows:

$${s}_{b} ( t ) = Acos ( 2 π ( {f}_{c} τ + \dot{f} τ {t}_{s} - \frac{f {\dot{τ}}^{2}}{2} ) , \qquad ( 2$$

wherein the instantaneous frequency of s_{b} is referred to as “beat frequency”, A denotes an amplitude of s_{b}, τ denotes a propagation delay or travelling time, and \dot{f} a chirp rate. The propagation delay in (2 can be rewritten in terms of distance R, and a relative velocity v of the target 250. After applying reasonable approximations, given the values that the variables take in practice, (2 can be approximated keeping only the terms that are dominant. So, s_{b} can be expressed as follows:

$${s}_{b} ( {t}_{s} , n ) ≅ Acos ( 2 π ( \frac{2 \dot{f} R}{c} {t}_{s} + \frac{2 {f}_{c} T v}{c} n ) + {F}_{0} ) , \qquad ( 3$$

wherein c denotes the speed of light.

As can be seen from (3, range and speed information can be separated since they appear as factors of different variables in (3. To determine the range information a Fourier Transform can be applied to s_{b} with respect to the fast-time variable tₛ, while the speed information can be recovered by applying the Fourier Transform to s_{b} with respect to the slow-time variable n.

In practice, the apparatus 100 can be applied to monitor a blood flow in the skin (of an animal or person). For this, a measurement depth of 1 mm, 2 mm, or up to 4 mm may be desired, where the blood vessels in the papillary dermis can be measured. Accordingly, the FMCW signal may be adapted such that it penetrates the skin up to 4 millimeters deep and that its reflections occurring 4 millimeters deep in the skin are measurable outside the skin. As well, a range resolution of approximately 0.1 mm may be desired.

To this end, the carrier frequency of the FMCW signal may be between 150 THz and 600 THz, a chirp bandwidth of the FMWC signal 112 may be between 1 THz and 10 THz, a chirping time of the FMCW signal may be between 5 μs and 100 μs, and/or a chirp repetition interval may be between 10 μs and 200 μs. Accordingly, a wavelength of the FMCW signal 112 may be between 500 nm and 2000 nm.

For example, a wavelength of about 900 nm, e.g., 976 nm, a bandwidth of approximately 3 THz, a carrier frequency of 307.2 THz, a number of chirps N of 1024, a chirping time of 9 μs, and a chirp repetition interval T of 20 μs have been found suitable. The skilled person will appreciate, though, that also other values of the aforementioned signal parameters of the FMCW signal 112 may be suitable, too.

Considering the above-mentioned signal parameters, different hardware may be used in implementations.

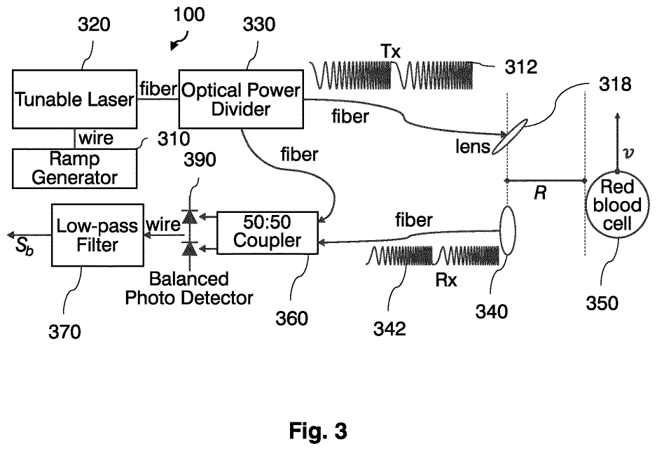

One option is to use a tunable Vertical-Cavity Surface-Emitting Laser (VCSEL) that, e.g., operates at a wavelength of (around) 1060 nm and may provide tunable wavelength range of nm, which allows a bandwidth of 3 THz. The VCSEL, in practice, may use Microelectromechanical Structures (MEMs) to achieve its tunable capability. The VCSEL may be optically pumped or electronically pumped, which facilitates a fabrication process so that costs may be less than for optically pumped VCSELs. An implementation utilizing a tunable laser is further explained with reference to FIG. 3 illustrating another embodiment of the apparatus 100.

In particular, FIG. 3 illustrates how the apparatus 100 may be implemented based on coherent optics.

As can be seen, the apparatus 100 may comprise a ramp generator 310 which drives a tunable laser 320. The tunable laser 320, e.g., a VCSEL, generates an FMCW signal 312. In the present example, the FMCW signal 312, e.g., is an FMCW laser signal. Optical fiber guides the FMCW laser signal 312 to an optical power divider 330. The optical power divider 330 splits up the FMCW laser signal 312 into separate parts. Also, it forwards a first part of the FMCW laser signal 312 to a 50:50 coupler 360 and a second part which is to be transmitted to a lens system 318 to form a desired beam of the FMCW laser signal 312. The lens system 318 is configured to emit the FMCW laser signal 312 at an angle (different from 90 degrees) such that it impinges a target 350 slantwise to determine a lateral velocity or speed. The present embodiment, e.g., serves for investigating a blood flow. Accordingly, the target 350 may be a flow of blood cells and the FMCW laser signal 312 may be emitted obliquely to a skin in order to measure a lateral speed/velocity of the flow.

The apparatus 100 further comprises another lens system 340 for receiving reflections 342 of the FMCW laser signal 312. In some embodiments, the apparatus 100 may further comprise a circulator to split a signal transmit and signal receive path of the apparatus 100 which allows to use a single lens system for transmission and reception.

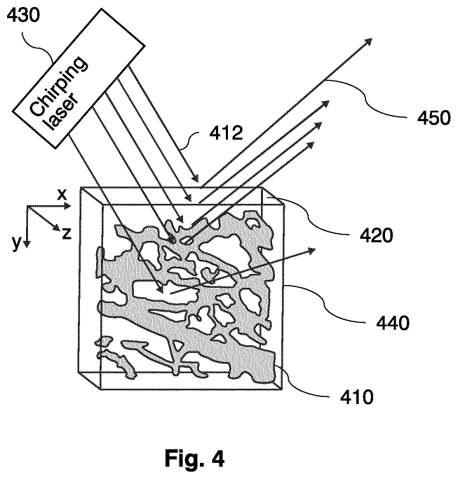

The received reflections 342 are forwarded to a 50:50 coupler 360 of the apparatus 100. The 50:50 coupler 360 combines the received reflections 342 with the FMCW laser signal 312 provided to the 50:50 coupler 360 to obtain a sum and difference signals. The sum and difference signals are then forwarded to a balanced photo detector 390. A photo current of the photo detector 390 is proportional to a power of the sum and difference signals respectively, which is the square of the field amplitude of the received reflections 342. So, a squaring operation provides a down-conversion from THz to the beat frequency in order to obtain the signal sp. The photo current, then, is forwarded to and filtered through a low-pass filter 370 to enable alias-free sampling, e.g., by an analog-to-digital converter (not shown). Then, range information, here particularly the distance R, and speed information, here particularly the speed/velocity v of the blood flow, can be determined from the signal sp. The distance and the speed/velocity, e.g., can be used for determining biomarkers and/or a physical condition of the cardiovascular system. In doing so, e.g., a relative distance of red blood cells may be determined. This, e.g., can be understood as “source localization analysis”. As well, the range information may allow a more accurate determination of the biomarkers and/or the physical condition, as laid out in more detail with reference to FIG. 4.

FIG. 4 schematically depicts a measurement of a body fluid 410 according to the present disclosure. In this example, the proposed concept is, e.g., applied in measuring a blood flow. To this end, a chirping laser 430 according to the present disclosure emits an FMCW signal 412 towards a perfused tissue 440. As indicated by arrows point towards the tissue 440, parts of the FMCW signal 412 penetrate the tissue 440 differently deep and reflections 450 of the FMCW signal 412 occur at different depths in the tissue as well as on a surface of the tissue 440, e.g., a skin surface 420. In practice, the FMCW signal 412, e.g., is particularly reflected by different blood vessels of different distance R. So, the reflections 450 have a different travelling time/distance and provide information on different blood vessels. Accordingly, considering the range information, e.g., reflections of blood vessels in different distances R can be distinguished. In doing so, e.g., information on the blood flow of different vessels in the tissue 440 may be distinguished by a respective distance R. In this way, e.g., the information on the blood flow of different types of vessels can be used to determine different, redundant, and/or complementary information. In practice, the information on the blood flow of different types of vessels, e.g., may provide information on different biomarkers and/or different attributes of the physical condition. As well, information on the blood flow of different types of vessels may complement and/or confirm each other, e.g., when determining a physical condition depending on various biomarkers.

So, one advantage of the proposed subject-matter is the ability to distinguish between different vessels of the body fluid. Different types of blood vessels (e.g. capillaries, veins, arteries) may deliver different information. Information on the blood flow in capillaries, in practice, contain information about endothelial activity, whereas veins and arteries are dominated by a cardiac signal. Accordingly, the processing circuit may be configured to extract information on blood flowing through capillaries and/or information on blood flowing through veins and/or arteries from the information on the body fluid in order to obtain information on a microvascular dynamics from the information on the blood flowing through the capillaries and/or obtain information on a cardiac activity from the information on the blood flowing through the veins and/or arteries.

Considering the microvascular dynamics, e.g., an endothelial functionality, regulatory effects of sphincter activation, and/or inflammation processes can be investigated. The information on the cardiac activity, e.g., indicates a health state and/or functional state of the heart.

The ability to separate information on body fluid in different vessels using a non-intrusive sensor may enable new medical applications for both physiological and psychological applications. In practice, e.g., it allows to estimate the blood pressure of a user or patient more accurately than, e.g., other devices using a fixed wavelength. In terms of psychological estimation, this would allow to measure more precisely a mental arousal of the user or patient.

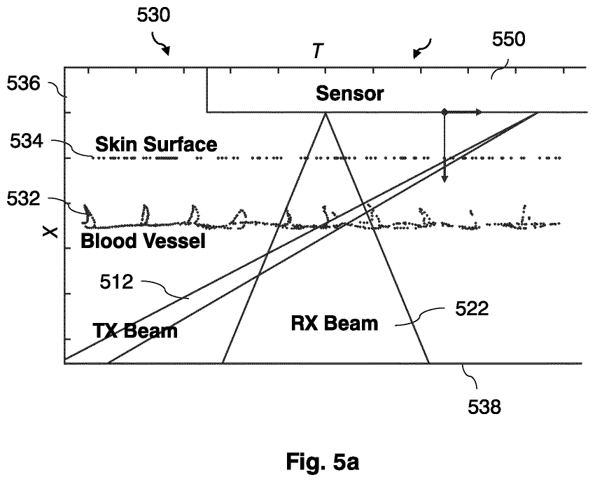

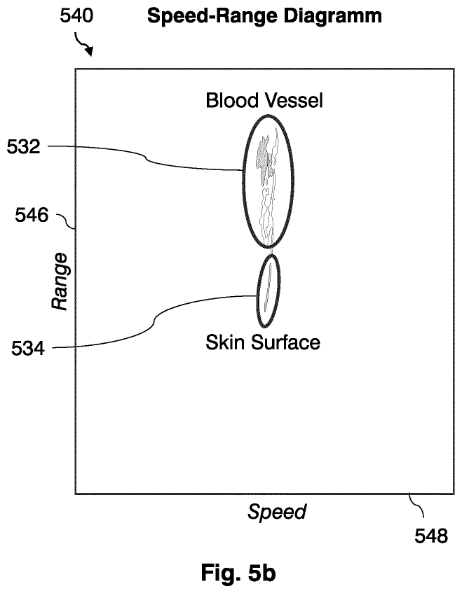

As well, the range information allows to remove or at least reduce motion artefacts or noise, as laid out in more detail with reference to FIGS. 5a and 5b.

FIG. 5a shows a diagram 530 which displays measurement samples 534 indicating a distance of reflections on the skin surface and measurement samples 532 indicating a distance of reflections on a blood vessel over time, wherein, with reference to the measurement samples 532 and 534, the ordinate 536 of the diagram 530 indicates the distance (the distance increases downwards) and the abscissa 538 indicates the time. Moreover, diagram 530 depicts a position of the receiver 550, here “sensor”, of the apparatus 100 and an emission beam 512 of the FMCW signal and a receiving area 522 of reflections, wherein, with reference to the emission and receiving area 512 and 522, the abscissa 538 indicates a lateral position.

As can be seen from the diagram 530, the sensor 550 is positioned in a certain distance to the skin surface and the emission beam 512 is emitted obliquely relative to the skin surface while the receiving area 522 is (approximately) perpendicular to the skin surface. In practice, e.g., the apparatus 100 exhibits a contact surface (not shown) that is supposed to have contact with the skin surface and the sensor 550 is placed in a certain distance to the contact surface.

It can also be seen from the measurement samples 532 and 534 differ in their distance such that the measurement samples 532 of the blood vessel can be clearly distinguished from measurement samples 534 of the skin surface. As mentioned above, measurements using the FMCW signal may provide information on the flow, as well. Accordingly, the measurement samples 532 and 534, e.g., are further associated with a velocity/speed of the flow, as shown in FIG. 5b.

FIG. 5b shows another diagram 540 displaying the speed of the measurement samples 532 and 534 with respect to their distance, wherein the ordinate 546 of diagram 540 indicates the distance (the distance increases upwards) and the abscissa 548 indicates the speed. As can be seen, the measurements samples 534 (in the lower circle) of the skin surface and the measurements samples 532 (in the upper circle) of the blood vessel differ in their distance as well as in their appearance or shape. The measurement samples 532 differ among themselves in their speed more than the measurement samples 534. Hence, the measurement samples 532 of the blood vessel are more broadly distributed than the measurement samples 534 of the skin surface. Plus, as can be seen in diagram 540, too, the distance of the measurement samples 532 is larger than the distance of the measurement samples 534 of the skin surface. So, in order to filter out noise resulting from reflections on the skin surface, the measurement samples 534 can be filtered out based on their difference to the measurement samples 532. In doing so, the measurement samples 534 of the skin surface, e.g., can be filtered out based on their distance and/or their speed. For this, e.g., appropriate algorithms can be used to distinguish between the measurement samples 532 and 534 based on the above differences. In embodiments, e.g., an appropriate segmentation algorithm may be used to distinguish between the measurement samples 532 and 534 in order to filter out noise from reflections on the skin surface. So, in practice, the processing circuit of the apparatus, e.g., may be configured to filter measurement data indicative of the reflections using the information on the depth, as stated above.

This, e.g., removes the requirement of a tight contact with and a fixed position of the measurement devices on the skin in order to avoid noise from movements relative to the skin. So, applying the above filtering of measurement data including relevant measurement samples (e.g., of a vessel) and irrelevant measurement samples (e.g., noise from reflections on the skin), e.g., allows ambulatory monitoring and a loose contact of the sensor or apparatus with the tissue to be investigated. In case, the apparatus is implemented in or as a wearable, this may make it more comfortable to wear the wearable as it does not have to be as tight against the skin as without the above filtering. The wearable, e.g., is a wristband, a chest strap, a (sports) watch, a finger clip, or the like. Also, the apparatus can be implemented in or as any other mobile device or a stationary device. In general, the proposed concept may be implemented in laser Doppler flowmetry or laser Doppler imaging applications.

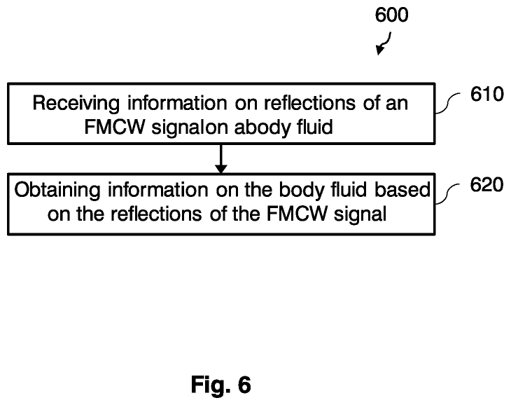

As the skilled person will appreciate, the proposed approach can be also implemented in a method and a respective computer program for executing the method, as laid out in more detail with reference to FIG. 6. The skilled person will appreciate that, in view of their technical relation, explanations in connection with the apparatus may also apply to features and aspects of the method and the computer program.

FIG. 6 shows a flow chart schematically illustrating an embodiment of a method 600 for noninvasive measurement of fluids in living tissue.

As can be seen, the method 600 comprises receiving 610 information on reflections of an FMCW signal on a body fluid. The method 600 further comprises obtaining 620 information on the body fluid based on the reflections of the FMCW signal. In doing so, the FMCW signal allows to determine a target distance for a more accurate investigation of the body fluid. As mentioned above, the FMCW signal provides that the information on the reflections, e.g., comprises information on a flow of the body fluid and, in particular, speed and range information the flow. As laid out above in more detail, the speed information, e.g., serves for determining biomarkers and/or a physical condition of the patient and/or his/her vessels. In doing so, the range information allows to distinguish between different vessels of the body fluid for a more accurate determination of the biomarkers and/or the physical condition, as laid out in more detail above.

The method 600, e.g., is carried out by a computer program having a program code for performing the method 600 when the computer program is executed on a processor or a programmable hardware. In general, the processor or programmable hardware can be any circuitry suitable for signal, information, and/or data processing. In practice, the program, e.g., is implemented in the processing circuit proposed herein.

### Further Examples Pertain to:

(1) An apparatus for non-invasive measurement of fluids in living tissue, the apparatus comprising:
   a frequency-modulated continuous wave, FMCW, transmitter configured to emit an FMCW signal for reflections of the FMCW signal on a body fluid;
   a receiver configured to receive the reflections of the FMCW signal; and
   a processing circuit configured to obtain information on the body fluid based on the reflections of the FMCW signal.
(2) The apparatus of (1), wherein the processing circuit is configured to obtain the information on the body fluid based on a comparison of the FMCW signal and its reflections.
(3) The apparatus of (1) or (2), wherein the information on the body fluid comprises information on a flow of the body fluid.
(4) The apparatus of (3), wherein the information on the flow comprises information on a flow velocity of the flow.
(5) The apparatus of any one of (1) to (4), wherein the information on the body fluid comprises information on a depth of the body fluid in the living tissue.
(6) The apparatus of (5), wherein the processing circuit is configured to filter measurement data indicative of the reflections using the information on the depth.
(7) The apparatus of any one of (1) to (6), wherein the processing circuit is configured to obtain information on vessel of the body fluid from the reflections of the FMCW signal.
(8) The apparatus of any one of (1) to (7), wherein the body fluid comprises blood.
(9) The apparatus of (8), wherein the information comprises information on a pulse rate, a blood pressure, and/or an oxygen saturation of the blood.
(10) The apparatus of (8) or (9), wherein the processing circuit is configured to extract information on blood flowing through capillaries and/or information on blood flowing through veins and/or arteries from the information on the body fluid.
(11) The apparatus of (10), wherein the processing circuitry is configured to obtain information on a microvascular dynamics from the information on the blood flowing through the capillaries.
(12) The apparatus of (10) or (11), wherein the processing circuitry is configured to obtain information on a cardiac activity from the information on the blood flowing through the veins and/or arteries.
(13) The apparatus of any one of (1) to (12), wherein the FMCW signal is adapted to penetrate skin up to 4 millimeters deep.
(14) The apparatus of any one of (1) to (13), wherein a carrier frequency of the FMCW signal is between 150 THz and 600 THz, a chirp bandwidth of the FMWC signal is between 1 THz and 10 THz, a chirping time of the FMCW signal is between 5 μs and 100 μs, and/or a chirp repetition interval is between 10 μs and 200 μs.
(15) The apparatus of any one of (1) to (14), wherein the FMCW transmitter comprises a Vertical-Cavity Surface-Emitting Laser, VCSEL.
(16) A wearable user device comprising an apparatus of any one of (1) to (15).
(17) A method for non-invasive measurement of fluids in living tissue, the method comprising:
   receiving information on reflections of a frequency-modulated continuous wave, FMCW, signal on a body fluid; and
   obtaining information on the body fluid based on the reflections of the FMCW signal.
(18) A computer program having a program code for performing a method according to (17) when the program is executed on a processor or a programmable hardware.

The aspects and features described in relation to a particular one of the previous examples may also be combined with one or more of the further examples to replace an identical or similar feature of that further example or to additionally introduce the features into the further example.

Examples may further be or relate to a (computer) program including a program code to execute one or more of the above methods when the program is executed on a computer, processor or other programmable hardware component. Thus, steps, operations or processes of different ones of the methods described above may also be executed by programmed computers, processors or other programmable hardware components. Examples may also cover program storage devices, such as digital data storage media, which are machine-, processor- or computer-readable and encode and/or contain machine-executable, processor-executable or computer-executable programs and instructions. Program storage devices may include or be digital storage devices, magnetic storage media such as magnetic disks and magnetic tapes, hard disk drives, or optically readable digital data storage media, for example. Other examples may also include computers, processors, control units, (field) programmable logic arrays ((F) PLAs), (field) programmable gate arrays ((F) PGAs), graphics processor units (GPU), application-specific integrated circuits (ASICs), integrated circuits (ICs) or system-on-a-chip (SoCs) systems programmed to execute the steps of the methods described above.

It is further understood that the disclosure of several steps, processes, operations or functions disclosed in the description or claims shall not be construed to imply that these operations are necessarily dependent on the order described, unless explicitly stated in the individual case or necessary for technical reasons. Therefore, the previous description does not limit the execution of several steps or functions to a certain order. Furthermore, in further examples, a single step, function, process or operation may include and/or be broken up into several sub-steps, -functions, -processes or -operations.

If some aspects have been described in relation to a device or system, these aspects should also be understood as a description of the corresponding method. For example, a block, device or functional aspect of the device or system may correspond to a feature, such as a method step, of the corresponding method. Accordingly, aspects described in relation to a method shall also be understood as a description of a corresponding block, a corresponding element, a property or a functional feature of a corresponding device or a corresponding system.

The following claims are hereby incorporated in the detailed description, wherein each claim may stand on its own as a separate example. It should also be noted that although in the claims a dependent claim refers to a particular combination with one or more other claims, other examples may also include a combination of the dependent claim with the subject matter of any other dependent or independent claim. Such combinations are hereby explicitly proposed, unless it is stated in the individual case that a particular combination is not intended. Furthermore, features of a claim should also be included for any other independent claim, even if that claim is not directly defined as dependent on that other independent claim.

1. An apparatus for non-invasive measurement of fluids in living tissue, the apparatus comprising:
a frequency-modulated continuous wave, FMCW, transmitter configured to emit an FMCW signal for reflections of the FMCW signal on a body fluid;
a receiver configured to receive the reflections of the FMCW signal; and
a processing circuit configured to obtain information on the body fluid based on the reflections of the FMCW signal.

2. The apparatus of claim 1, wherein the processing circuit is configured to obtain the information on the body fluid based on a comparison of the FMCW signal and its reflections.

3. The apparatus of claim 1, wherein the information on the body fluid comprises information on a flow of the body fluid.

4. The apparatus of claim 3, wherein the information on the flow comprises information on a flow velocity of the flow.

5. The apparatus of claim 1, wherein the information on the body fluid comprises information on a depth of the body fluid in the living tissue.

6. The apparatus of claim 5, wherein the processing circuit is configured to filter measurement data indicative of the reflections using the information on the depth.

7. The apparatus of claim 1, wherein the processing circuit is configured to obtain information on vessel of the body fluid from the reflections of the FMCW signal.

8. The apparatus of claim 1, wherein the body fluid comprises blood.

9. The apparatus of claim 8, wherein the information comprises information on a pulse rate, a blood pressure, and/or an oxygen saturation of the blood.

10. The apparatus of claim 8, wherein the processing circuit is configured to extract information on blood flowing through capillaries and/or information on blood flowing through veins and/or arteries from the information on the body fluid.

11. The apparatus of claim 10, wherein the processing circuitry is configured to obtain information on a microvascular dynamics from the information on the blood flowing through the capillaries.

12. The apparatus of claim 10, wherein the processing circuitry is configured to obtain information on a cardiac activity from the information on the blood flowing through the veins and/or arteries.

13. The apparatus of claim 1, wherein the FMCW signal is adapted to penetrate skin up to 4 millimeters deep.

14. The apparatus of claim 1, wherein a carrier frequency of the FMCW signal is between 150 THz and 600 THz, a chirp bandwidth of the FMWC signal is between 1 THz and THz, a chirping time of the FMCW signal is between 5 μs and 100 μs, and/or a chirp repetition interval is between 10 μs and 200 μs.

15. The apparatus of claim 1, wherein the FMCW transmitter comprises a Vertical-Cavity Surface-Emitting Laser, VCSEL.

16. A wearable user device comprising an apparatus of claim 1.

17. A method for non-invasive measurement of fluids in living tissue, the method comprising:
receiving information on reflections of a frequency-modulated continuous wave, FMCW, signal on a body fluid; and
obtaining information on the body fluid based on the reflections of the FMCW signal.

18. A computer program having a program code for performing a method according to claim 17 when the program is executed on a processor or a programmable hardware.
